(19) United States
(12) Patent Application Publication
(10) Pub. No.: US 2021/0311030 A1
(43) Pub. Date: Oct. 7, 2021

(54) FLUORESCENT-LABELED ANTIBODY OR ANTIBODY FRAGMENT

(71) Applicant: TOKYO INSTITUTE OF TECHNOLOGY, Tokyo (JP)

(72) Inventors: Shinichi SATO, Tokyo (JP); Hiroyuki NAKAMURA, Tokyo (JP); Hiroshi UEDA, Tokyo (JP)

(73) Assignee: TOKYO INSTITUTE OF TECHNOLOGY, Tokyo (JP)

(21) Appl. No.: 17/264,722

(22) PCT Filed: Jul. 10, 2019
(86) PCT No.: PCT/JP2019/027279

§ 371 Date: Jan. 29, 2021

(30) Foreign Application Priority Data
Jul. 30, 2018 (JP) 2018-142089

(51) Int. Cl. G01N 33/533; G01N 33/542

(52) U.S. Cl. CPC G01N 33/533; G01N 33/542

(57) ABSTRACT

Provided is an antibody or antibody fragment labeled with a fluorescent dye having the following properties (1) and (2):
(1) the fluorescent dye is quenched in the absence of an antigen and the quenching of the fluorescent dye is cancelled in the presence of an antigen; and
(2) the fluorescent dye is contained in a compound binding to an amino acid residue in the antibody or antibody fragment.

### TECHNICAL FIELD

The present invention relates to a fluorescence-labeled antibody or antibody fragment. The fluorescence-labeled antibody or antibody fragment enables detection of an antigen by a change in fluorescence intensity, and therefore can be used for diagnostic agents for disease, or the like.

### BACKGROUND ART

Sato, one of the present inventors, has recently developed a technique for specifically chemically modifying a tyrosine residue in a complementarity determining region while retaining the antigen recognition ability of an antibody (Non Patent Literature 1). On the other hand, Ueda, one of the present inventors, has developed a molecule in which a protein having a structure of a variable region site of an antibody is conjugated with a fluorescent molecule. The molecule, which is named Q-body, is a turn-on-type fluorescence sensor in which the fluorescence of the fluorescent molecule is quenched in the absence of an antigen, and the quenching is cancelled in the presence of an antigen (Non Patent Literature 2 and Patent Literatures 1 and 2).

### CITATION LIST

### Patent Literature

[Patent Literature 1] Japanese Patent No. 5043237
[Patent Literature 2] International Publication No. WO 2013/065314

### Non Patent Literature

[Non Patent Literature 1] Masaki Matsumura, Shinichi Sato and Hiroyuki Nakamura, “Study on Reaction Conditions for Protein Functionalization by Tyrosine Residue Labeling”, the Chemical Society of Japan, the 98th Annual Meeting in Spring (2018), Lecture proceedings
[Non Patent Literature 2] R. Abe, H. Ohashi, I. Iijima, M. Ihara, H. Takagi, T. Hohsaka and H. Ueda ““Quenchbodies”: quench-based antibody probes that show antigen-dependent fluorescence” J. Am. Chem. Soc. 133, 17386-17394 (2011).

### SUMMARY OF INVENTION

### Technical Problem

Q-body enables homogeneous antigen detection which does not require an immobilization or washing step, and Q-body is used in various fields such as clinical diagnosis and environmental analysis. However, a protein synthesis system of a cell-free system is needed for efficiently preparing Q-body, and application to high-volume synthesis is a challenge.

The present invention has been made under the above-mentioned circumstances, and an object of the present invention is to provide means which enables high-volume supply of Q-body.

### Solution to Problem

The present inventors have found that by modifying an antibody with a fluorescent dye using the technique for chemically modifying a complementarity determining region of an antibody, the fluorescent dye is quenched in the absence of an antigen and the quenching of the fluorescent dye is cancelled in the presence of an antigen as with Q-body. In addition, the present inventors have found that when as the fluorescent dye used here, a fluorescent dye having 4,4-difluoro-4-bora-3a,4a-diaza-s-indacene (BODIPY) as a basic skeleton is used, the antigen detection sensitivity is improved. Further, the present inventors have also found that modification of an antibody with a fluorescent dye can be performed by an electrochemical method. The present invention has been completed based on these findings.

That is, the present invention provides the following [1] to [18].

[1] A fluorescence-labeled antibody or antibody fragment which is an antibody or antibody fragment labeled with a fluorescent dye, the fluorescent dye having the following properties (1) and (2):
(1) the fluorescent dye is quenched in the absence of an antigen and the quenching of the fluorescent dye is cancelled in the presence of an antigen; and
(2) the fluorescent dye is contained in a compound binding to an amino acid residue in the antibody or antibody fragment.
[2] The fluorescence-labeled antibody or antibody fragment according to [1], wherein the compound binding to an amino acid residue in the antibody or antibody fragment is a compound represented by any of general formulas (I) to (VIII):

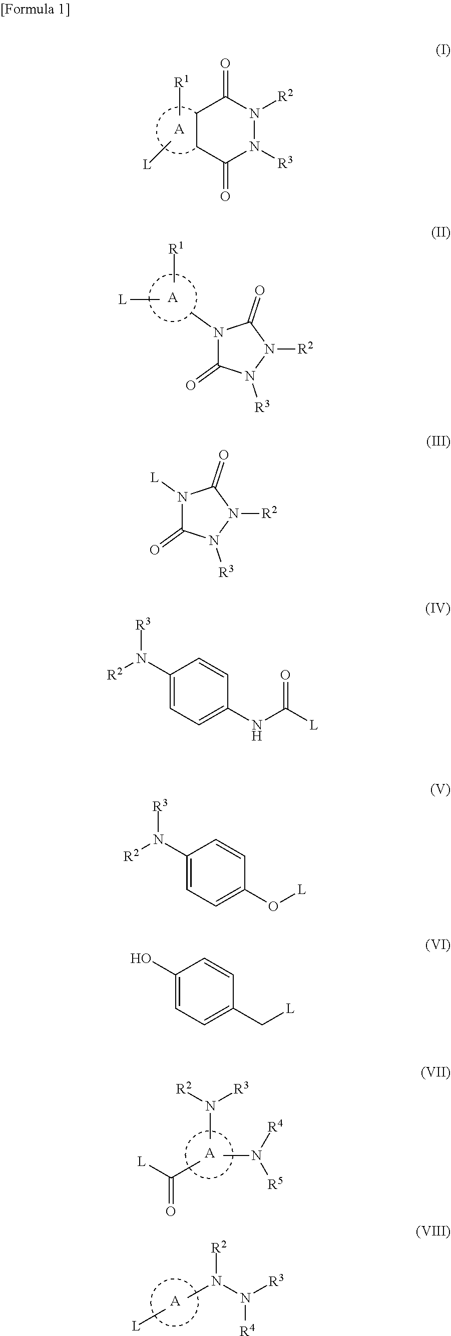

wherein A represents a conjugated ring, L represents a linker having a fluorescent dye at an end (provide that L can be present at any position on A when L is present on A), R¹ represents a hydrogen atom, or represents one or two amino groups, acetamide groups, hydroxy groups, alkyl groups or alkoxy groups each present at any position on A, and R², R³, R⁴ and R⁵ each represent a hydrogen atom, an alkyl group, or an aromatic group optionally having a substituent.
[3] The fluorescence-labeled antibody or antibody fragment according to [1], wherein the compound binding to an amino acid residue in the antibody or antibody fragment is a compound binding to a tyrosine residue in the antibody or antibody fragment.
[4] The fluorescence-labeled antibody or antibody fragment according to [3], wherein the compound binding to a tyrosine residue in the antibody or antibody fragment is a compound represented by any of general formulas (I) to (VI):

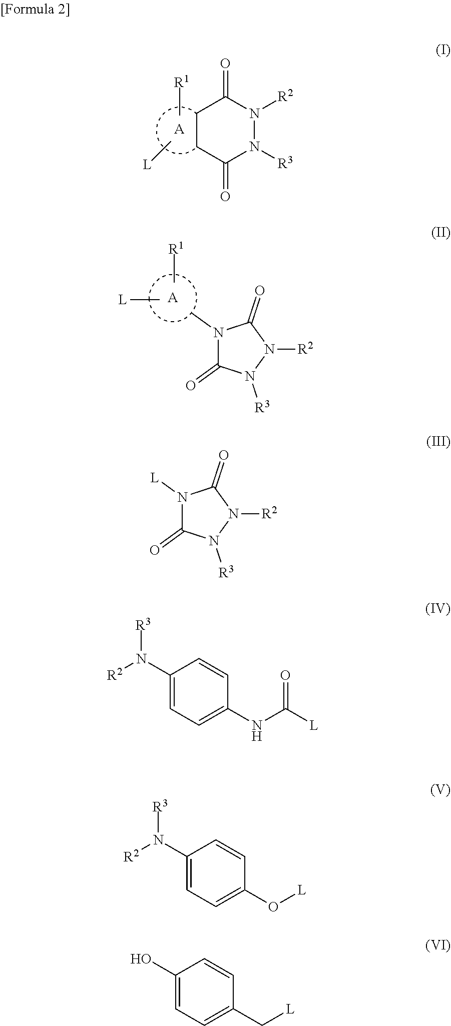

wherein A represents a conjugated ring, L represents a linker having a fluorescent dye at an end (provide that L can be present at any position on A when L is present on A), R¹ represents a hydrogen atom, or represents one or two amino groups, acetamide groups, hydroxy groups, alkyl groups or alkoxy groups each present at any position on A, and R² and R³ each represent a hydrogen atom, an alkyl group, or an aromatic group optionally having a substituent.
[5] The fluorescence-labeled antibody or antibody fragment according to [3], wherein the compound binding to a tyrosine residue in the antibody or antibody fragment is a compound represented by general formula (Ia):

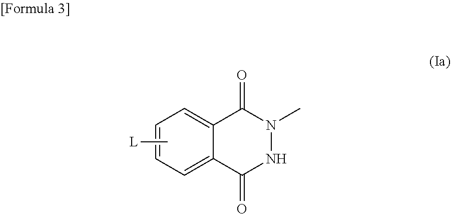

wherein L represents a linker which is present at any position on the benzene ring and has a fluorescent dye at an end.
[6] The fluorescence-labeled antibody or antibody fragment according to any one of [1] to [5], wherein the fluorescent dye is a fluorescent dye having 4,4-difluoro-4-bora-3a,4a-diaza-s-indacene as a basic skeleton.
[7] The fluorescence-labeled antibody or antibody fragment according to any one of [1] to [6], wherein the antibody or antibody fragment contains a light chain and a heavy chain, and the fluorescent dye binds to a variable region of the light chain or the heavy chain.
[8] The fluorescence-labeled antibody or antibody fragment according to [7], wherein the antibody or antibody fragment binds to a quencher for the fluorescent dye, and the quencher binds to a variable region of a chain different from the chain to which the fluorescent dye binds.
[9] The fluorescence-labeled antibody or antibody fragment according to [7], wherein the antibody or antibody fragment binds to another fluorescent dye forming a FRET pair with the fluorescent dye, and the other fluorescent dye binds to a variable region of a chain different from the chain to which the fluorescent dye binds.
[10] The fluorescence-labeled antibody or antibody fragment according to any one of [1] to [9], wherein the antibody or antibody fragment is an antibody or antibody fragment against a substance serving as a marker for disease.
[11] A diagnostic agent for disease, comprising the fluorescence-labeled antibody or antibody fragment according to [10].
[12] A method for producing a fluorescence-labeled antibody or antibody fragment, the method comprising steps (1) and (2):
(1) reacting a compound binding to an amino acid residue in an antibody or antibody, the compound containing a functional group for use in click reaction, with the antibody or antibody fragment to bind the compound to the amino acid residue in the antibody or antibody fragment; and
(2) reacting a fluorescent dye with the functional group for use in click reaction in the compound binding to an amino acid residue in an antibody or antibody fragment, to bind the fluorescent dye to the functional group.
[13] The method for producing a fluorescence-labeled antibody or antibody fragment according to [12], wherein the compound binding to an amino acid residue in an antibody or antibody fragment is a compound represented by any of general formulas (Ib) to (VIIIb):

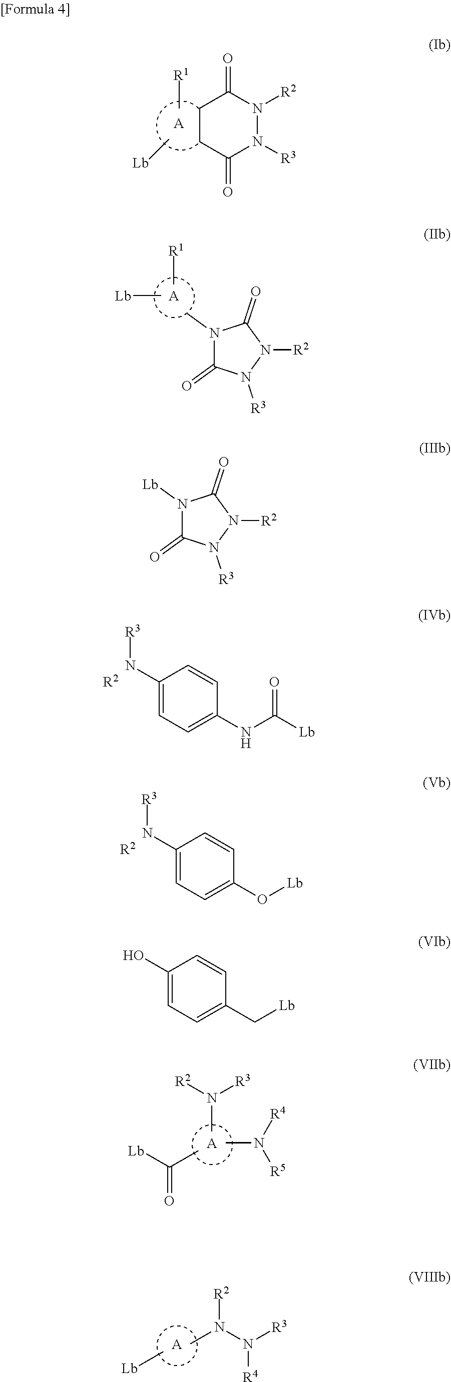

wherein A represents a conjugated ring, Lb represents a linker having a functional group for use in click reaction at an end (provide that Lb can be present at any position on A when Lb is present on A), R¹ represents a hydrogen atom, or represents one or two amino groups, acetamide groups, hydroxy groups, alkyl groups or alkoxy groups each present at any position on A, and R², R³, R⁴ and R⁵ each represent a hydrogen atom, an alkyl group, or an aromatic group optionally having a substituent.
[14] The method for producing a fluorescence-labeled antibody or antibody fragment according to [12], wherein the compound binding to an amino acid residue in an antibody or antibody fragment is a compound binding to a tyrosine residue in the antibody or antibody fragment.
[15] The method for producing a fluorescence-labeled antibody or antibody fragment according to [14], wherein the compound binding to a tyrosine residue in the antibody or antibody fragment is a compound represented by any of general formulas (Ib) to (VIb):

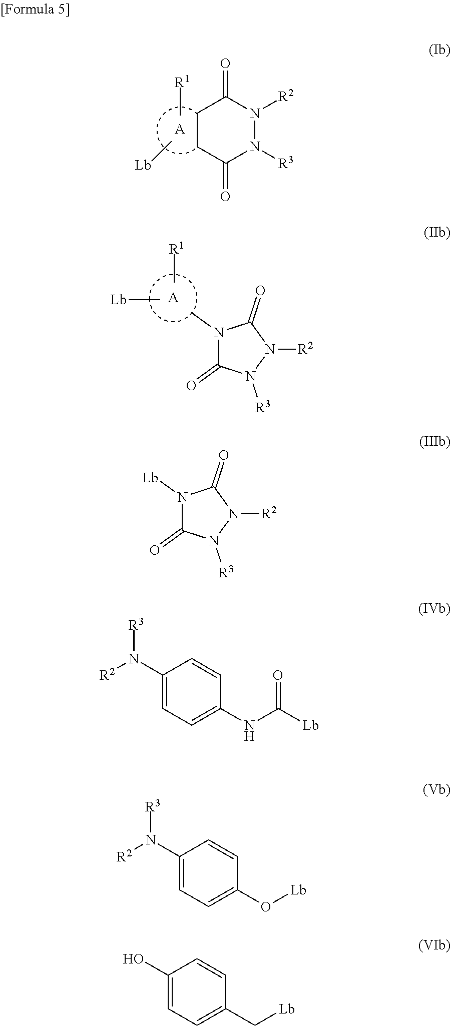

wherein A represents a conjugated ring, Lb represents a linker having a functional group for use in click reaction at an end (provide that Lb can be present at any position on A when Lb is present on A), R¹ represents a hydrogen atom, or represents one or two amino groups, acetamide groups, hydroxy groups, alkyl groups or alkoxy groups each present at any position on A, and R² and R³ each represent a hydrogen atom, an alkyl group, or an aromatic group optionally having a substituent.
[16] The method for producing a fluorescence-labeled antibody or antibody fragment according to [14], wherein the compound binding to a tyrosine residue in the antibody or antibody fragment is a compound represented by general formula (Iab):

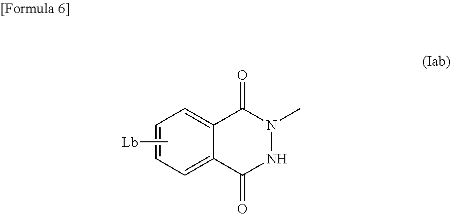

wherein Lb represents a linker which is present at any position on the benzene ring and has a functional group for use in click reaction at an end.
[17] The method for producing a fluorescence-labeled antibody or antibody fragment according to any one of [12] to [16], wherein the fluorescent dye is a fluorescent dye having 4,4-difluoro-4-bora-3a,4a-diaza-s-indacene as a basic skeleton.
[18] The method for producing a fluorescence-labeled antibody or antibody fragment according to any one of [12] to [17], wherein step (1) is a step of applying a voltage to a solution containing the compound binding to an amino acid residue in an antibody or antibody fragment and the antibody or antibody fragment.

The present description includes the contents disclosed in the description and/or the drawings of Japanese Patent Application No. 2018-142089 based on which the priority to the present application is claimed.

### Advantageous Effect of Invention

The present invention provides a novel fluorescence-labeled antibody or antibody fragment. The fluorescence-labeled antibody or antibody fragment enables detection of an antigen by a change in fluorescence intensity, and therefore can be used for diagnostic agents for disease, or the like.

### BRIEF DESCRIPTION OF DRAWINGS

FIG. 1 shows a fluorescence spectrum of DBCO-PEG₄-TAMRA bound to rituximab in the presence or absence of a denaturant.

FIG. 2 shows a fluorescence spectrum of DBCO-PEG₄-TAMRA which is not bound to rituximab in the presence or absence of a denaturant.

FIG. 3 shows fluorescence spectra of DBCO-PEG₄-TAMRA bound to rituximab in the presence of cell lysates. As the cell lysates, a lysate of cells which express an antigen of rituximab (left) and a lysate of cells which do not express an antigen of rituximab (right) are used.

FIG. 4 shows fluorescence spectra of DBCO-TAMRA bound to rituximab in the presence of cell lysates. As the cell lysates, a lysate of cells which express an antigen of rituximab (left) and a lysate of cells which do not express an antigen of rituximab (right) are used.

FIG. 5 shows structures, excitation wavelengths and fluorescence wavelengths of fluorescent dyes used in Example 3.

FIG. 6 schematically shows a method for electrochemically modifying a protein.

FIG. 7 shows fluorescence spectra of fluorescence-labeled rituximab prepared by an electrochemical method, where spectra in the presence of a denaturant (denatured) and in the absence of a denaturant (in PBS) are shown.

### DESCRIPTION OF EMBODIMENTS

Hereinafter, the present invention will be described in detail.

### (A) Definition

In the present invention, the “alkyl group having 1 to 20 carbon atoms” is a linear or branched alkyl group having 1 or more and 20 or less carbon atoms, and examples thereof include a methyl group, an ethyl group, a n-propyl group, an iso-propyl group, a n-butyl group, an iso-butyl group, a sec-butyl group, a tert-butyl group, a pentyl group, an iso-pentyl group, a neo-pentyl group, a hexyl group, an iso-hexyl group, a heptyl group, an iso-heptyl group, an octyl group, a nonyl group, a decyl group, an undecyl group, a dodecyl group, a tridecyl group, a tetradecyl group, a pentadecyl group, a hexadecyl group, a heptadecyl group, an octadecyl group, a nonadecyl group and an icosyl group.

In the present invention, the “alkyl group having 1 to 10 carbon atoms” is a linear or branched alkyl group having 1 or more and 10 or less carbon atoms, and examples thereof include a methyl group, an ethyl group, a n-propyl group, an iso-propyl group, a n-butyl group, an iso-butyl group, a sec-butyl group, a tert-butyl group, a pentyl group, an iso-pentyl group, a neo-pentyl group, a hexyl group, an iso-hexyl group, a heptyl group, an iso-heptyl group, an octyl group, a nonyl group and a decyl group.

In the present invention, the “alkyl group having 1 to 3 carbon atoms” is a linear or branched alkyl group having 1 or more and 3 or less carbon atoms, and examples thereof include a methyl group, an ethyl group, a n-propyl group and an iso-propyl group.

In the present invention, the “alkoxy group having 1 to 20 carbon atoms” is a linear or branched alkoxy group having 1 or more and 20 or less carbon atoms, and examples thereof include a methoxy group, an ethoxy group, a n-propoxy group, an iso-propoxy group, a n-butoxy group, an iso-butoxy group, a sec-butoxy group, a tert-butoxy group, a pentyloxy group, an iso-pentyloxy group, a neo-pentyloxy group, a hexyloxy group, an iso-hexyloxy group, a heptyloxy group, an iso-heptyloxy group, an actyloxy group, a nonyloxy group, a decyloxy group, an undecyloxy group, a dodecyloxy group, a tridecyloxy group, a tetradecyloxy group, a pentadecyloxy group, a hexadecyloxy group, a heptadecyloxy group, an octadecyloxy group, a nonadecyloxy group and an icosyloxy group.

In the present invention, the “alkoxy group having 1 to 10 carbon atoms” is a linear or branched alkoxy group having 1 or more and 10 or less carbon atoms, and examples thereof include a methoxy group, an ethoxy group, a n-propoxy group, an iso-propoxy group, a n-butoxy group, an iso-butoxy group, a sec-butoxy group, a tert-butoxy group, a pentyloxy group, an iso-pentyloxy group, a neo-pentyloxy group, a hexyloxy group, an iso-hexyloxy group, a heptyloxy group, an iso-heptyloxy group, an actyloxy group, a nonyloxy group and a decyloxy group.

In the present invention, the “alkoxy group having 1 to 3 carbon atoms” is a linear or branched alkoxy group having 1 or more and 3 or less carbon atoms, and examples thereof include a methoxy group, an ethoxy group, a n-propoxy group and an iso-propoxy group.

In the present invention, the “aromatic group optionally having a substituent” means an aromatic group having no substituent or an aromatic group having at least one substituent. Here, the “aromatic group” is a group obtained by removing one hydrogen atom from an aromatic compound, and examples thereof include a phenyl group, a 1-naphthyl group, a 2-naphthyl group, a pyridine-2-yl group, a pyridine-3-yl group, a pyridine-4-yl group, a pyrimidine-2-yl group, a pyrimidine-4-yl group, a pyrimidine-5-yl group, a pyrazine-2-yl group, a pyrazine-3-yl group, a pyridazine-3-yl group, a pyridazine-4-yl group, a furan-2-yl group, a furan-3-yl group, a thiophene-2-yl group, a thiophene-3-yl group, a pyrrole-1-yl group, a pyrrole-2-yl group, a pyrrole-3-yl group, a pyrazole-1-yl group, a pyrazole-3-yl group, a pyrazole-4-yl group, a pyrazole-5-yl group, an imidazole-1-yl group, an imidazole-2-yl group, an imidazole-4-yl group and an imidazole-5-yl group. The substituent can be selected from the group consisting of a methyl group, a fluorine atom, a chlorine atom, a bromine atom, a hydroxy group, a methoxy group and the like. Preferred examples of the aromatic group having at least one substituent include phenyl groups having at least one substituent, and specific examples thereof include a 2-methylphenyl group, a 3-methylphenyl group, a 4-methylphenyl group, a 2,3-dimethylphenyl group, a 2,4-dimethylphenyl group, a 2,5-dimethylphenyl group, a 2,6-dimethylphenyl group, a 3,4-dimethylphenyl group, a 3,5-dimethylphenyl group, a 2-fluorophenyl group, a 3-fluorophenyl group, a 4-fluorophenyl group, a 2,3-difluorophenyl group, a 2,4-difluorophenyl group, a 2,5-difluorophenyl group, a 2,6-difluorophenyl group, a 3,4-difluorophenyl group, a 3,5-difluorophenyl group, a 2-chlorophenyl group, a 3-chlorophenyl group, a 4-chlorophenyl group, a 2,3-dichlorophenyl group, a 2,4-dichlorophenyl group, a 2,5-dichlorophenyl group, a 2,6-dichlorophenyl group, a 3,4-dichlorophenyl group, a 3,5-dichlorophenyl group, a 2-bromophenyl group, a 3-bromophenyl group, a 4-bromophenyl group, a 2,3-dibromophenyl group, a 2,4-dibromophenyl group, a 2,5-dibromophenyl group, a 2,6-dibromophenyl group, a 3,4-dibromophenyl group, a 3,5-dibromophenyl group, a 2-hydroxyphenyl group, a 3-hydroxyphenyl group, a 4-hydroxyphenyl group, a 2-methoxyphenyl group, a 3-methoxyphenyl group and a 4-methoxyphenyl group.

In the present invention, the “functional group for use in click reaction” is, for example, an azide group or an ethynyl group.

In the present invention, the “conjugated ring” is a ring having a conjugated double bond. The conjugated ring may be either an aromatic ring or a nonaromatic ring. The conjugated ring may be either a ring composed only of carbon atoms, or a heterocyclic ring containing an atom other than carbon, for example an atom such as nitrogen, oxygen or sulfur. Specific examples of the conjugated ring include six-membered rings such as a benzene ring, a 1,3-cyclohexadiene ring, a pyridine ring, a pyrimidine ring, a pyrazine ring, a pyridazine ring and a triazine ring; and five-membered rings such as a cyclopentadiene ring, a furan ring, a thiophene ring, a pyrrole ring, a pyrazole ring and an imidazole ring.

### (B) Fluorescence-Labeled Antibody or Antibody Fragment

The fluorescence-labeled antibody or antibody fragment of the present invention is labeled with a fluorescent dye. This fluorescent dye is quenched in the absence of an antigen and the quenching is cancelled in the presence of an antigen as with a fluorescent dye in Q-body. Since such quenching and cancellation thereof are due to interaction with tryptophane residues present in a heavy-chain variable region and a light-chain variable region of the antibody, it is preferable that the fluorescent dye be present at a position allowing the fluorescent dye to interact with the tryptophane residues. Specifically, the fluorescent dye binds through the linker to preferably a variable region of a heavy chain or a light chain in the antibody or antibody fragment, more preferably a complementarity determining region of a heavy chain or a light chain in the antibody or antibody fragment. It has been heretofore considered that when a fluorescent dye is bound to the complementarity determining region, the antigen recognition ability is lost because the complementarity determining region is involved in the antigen recognition ability, but the present inventors have confirmed that the antigen recognition ability is not lost.

The fluorescent dye is contained in a compound binding to an amino acid residue in the antibody or antibody fragment, and when such a compound binds to an amino acid residue in the antibody or antibody fragment, the antibody or antibody fragment is labeled with the fluorescent dye.

Examples of the compound binding to an amino acid residue in an antibody or antibody fragment include compounds represented by general formulas (I) to (VIII).

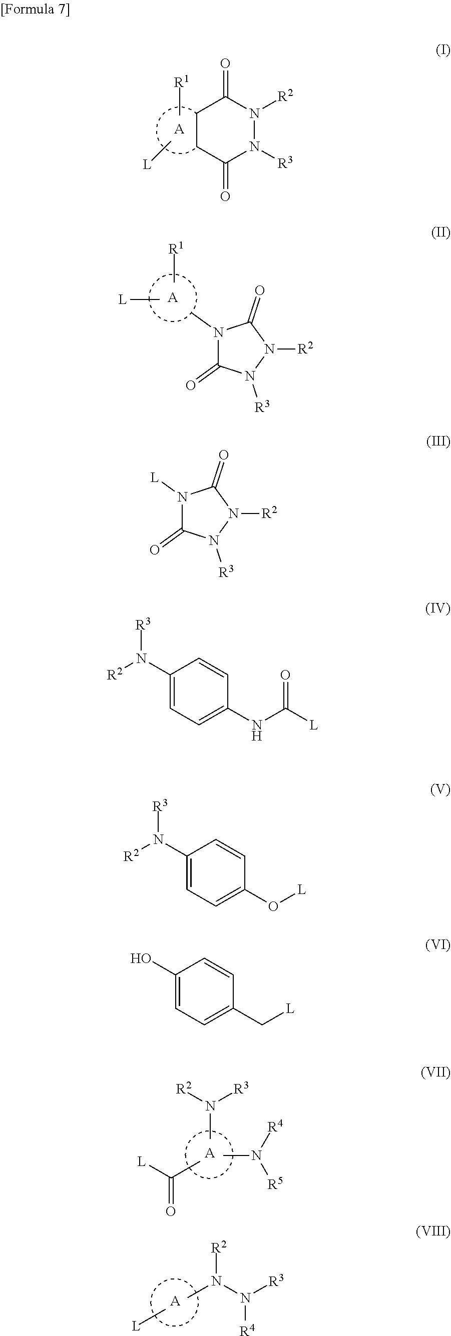

In general formulas (I), (II), (VII) and (VIII), A represents a conjugated ring. A may be a conjugated ring, and is preferably a benzene ring in general formulas (I), (II) and (VII), and preferably a pyridine ring in general formula (VIII).

In general formulas (I) and (II), R¹ represents a hydrogen atom, or represents one or two amino groups, acetamide groups, hydroxy groups, alkyl groups or alkoxy groups each present at any position on A. Here, the number of carbons in each of the alkyl group and the alkoxy group is not particularly limited, and is preferably 1 to 20, more preferably 1 to 10, especially preferably 1 to 3. R¹ may be a group as described above, and is preferably a hydrogen atom, or one amino group, an acetamide group or a methoxy group, more preferably a hydrogen atom or one methoxy group. When the number of the substituents present is 2, the substituents may be the same or different. The substituents can be each present at any position on the conjugated ring, and from the viewpoint of ease of synthesis, it is preferable that the substituents be each present at a position away from the adjacent heterocyclic ring.

In general formulas (I), (II), (III), (IV), (V), (VII) and (VIII), R² represents a hydrogen atom, an alkyl group, or an aromatic group optionally having a substituent. Here, the number of carbon atoms in the alkyl group is not particularly limited, and is preferably 1 to 20, more preferably 1 to 10, especially preferably 1 to 3. R² may be a group as described above, and is preferably a hydrogen atom, a methyl group or a phenyl group, more preferably a methyl group in general formula (I), preferably a hydrogen atom or a methyl group, more preferably a methyl group in general formula (II), preferably a hydrogen atom, a methyl group, a phenyl group, a phenyl group having a methoxy group, or a phenyl group having a fluorine atom, more preferably a methyl group in general formula (III), preferably a hydrogen atom or a methyl group in general formulas (IV), (V) and (VII), and preferably a hydrogen atom in the general formula (VIII).

In general formulas (I), (II), (III), (IV), (V), (VII) and (VIII), R³ represents a hydrogen atom, an alkyl group, or an aromatic group optionally having a substituent. Here, the number of carbon atoms in the alkyl group is not particularly limited, and is preferably 1 to 20, more preferably 1 to 10, especially preferably 1 to 3. R³ may be a group as described above, and is preferably a hydrogen atom in general formulas (I), (II) and (III), preferably a hydrogen atom or a methyl group in general formulas (IV) and (V), and preferably a hydrogen atom in general formulas (VII) and (VIII).

In general formulas (VII) and (VIII), R⁴ represents a hydrogen atom, an alkyl group, or an aromatic group optionally having a substituent. Here, the number of carbon atoms in the alkyl group is not particularly limited, and is preferably 1 to 20, more preferably 1 to 10, especially preferably 1 to 3. R⁴ may be a group as described above, and is preferably a hydrogen atom or a methyl group in general formula (VII), and preferably a hydrogen atom in general formula (VIII).

In general formula (VII), R⁵ represents a hydrogen atom, an alkyl group, or an aromatic group optionally having a substituent. Here, the number of carbon atoms in the alkyl group is not particularly limited, and is preferably 1 to 20, more preferably 1 to 10, especially preferably 1 to 3. R⁵ may be a group as described above, and is preferably a hydrogen atom.

In general formulas (I), (II), (III), (IV), (V), (VI), (VII) and (VIII), L represents a linker having a fluorescent dye at an end. L can be present at any position on A when L is present on A. The linker may have any structure as long as the structure does not cause the fluorescent dye to lose its function, and the linker is preferably an alkylene group. One or more —CH₂— in the alkylene group may be substituted with —O—, —S—, —NH— or —CO—. In the present invention, normally the fluorescent dye is bound through click reaction, and therefore —CH₂— may be substituted with a 1H-1,2,3-triazole-1,4-diyl group generated by click reaction (reaction of an azide group with an ethynyl group). The number of carbon atoms in the alkylene group is not particularly limited, and is preferably 5 to 20, more preferably 5 to 15. When —CH₂— in the alkylene group is substituted with another group, the other group is considered to contribute to the “number of carbon atoms in the alkylene group” as a group having one carbon atom.

The compounds binding to an amino acid residue in the antibody or antibody fragment are not limited to the compounds represented by general formulas (I) to (VIII) described above, and include, for example, the following compounds.

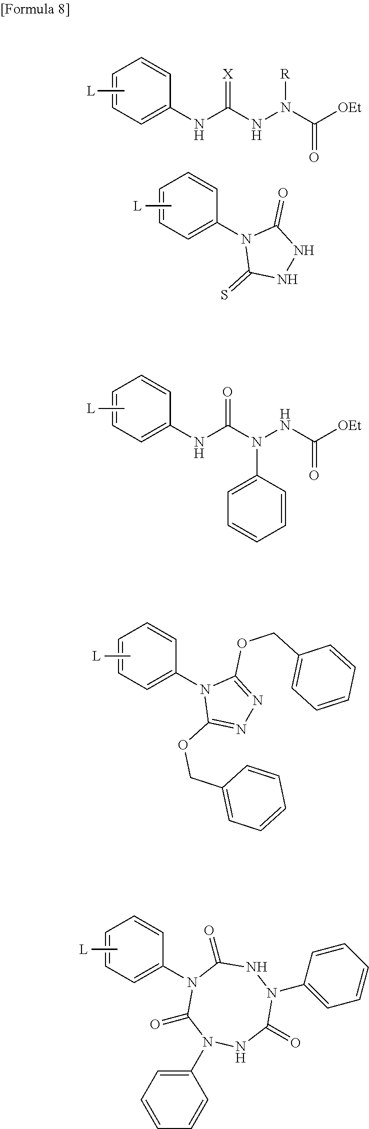

In the compounds, L has the same meaning of L in general formulas (I) to (VIII), X represents an oxygen atom or a sulfur atom, R represents a hydrogen atom or a methyl group when X represents an oxygen atom, and R represents a hydrogen atom when X represents a sulfur atom.

Among the compounds binding to an amino acid residue in the antibody or antibody fragment, compounds binding to a tyrosine residue in the antibody or antibody fragment are preferable. The compounds represented by general formulas (I) to (VI) can all bind to a tyrosine residue. That is, the compound represented by general formula (I) (Japanese Patent Laid-Open No. 2016-108266 and WO 2017/061288) binds to a tyrosine residue, and the compounds represented by general formulas (II) and (III) (S. Sato et al., Chem. Commun. 54, 5871-5874 (2018)) bind to a tyrosine residue. The compound represented by general formula (IV) (S. Sato et al., Angew. Chem. Int. Ed. 52, 8681-8684 (2013)), the compound represented by general formula (V) (K. L. Seim et al., J. Am. Chem. Soc. 133, 16970-16976 (2011)), and the compound represented by general formula (VI) (J. S. Rees et al., Mol Cell proteomics. 14(11): 2848-2856 (2015)) bind to electron-rich amino acid residues such as a tyrosine residue, a tryptophane residue, a free cysteine residue and a histidine residue.

As described above, the compounds represented by general formulas (I) to (III) bind specifically to a tyrosine residue in the antibody or antibody fragment, and the compounds represented by general formulas (VI) to (VI) can bind to a tyrosine residue in the antibody or antibody fragment. Since the complementarity determining region of the antibody has a large number of tyrosine residues exposed on the surface, the fluorescent dye can be bound specifically to the complementarity determining region of the antibody or antibody fragment by using the compounds represented by general formulas (I) to (VI).

As the compound binding to a tyrosine residue in the antibody or antibody fragment, any of the compounds represented by general formulas (I) to (VI) may be used, and it is preferable to use compounds represented by general formula (I). Among the compounds represented by general formula (I), compounds represented by general formula (Ia) are preferable to use.

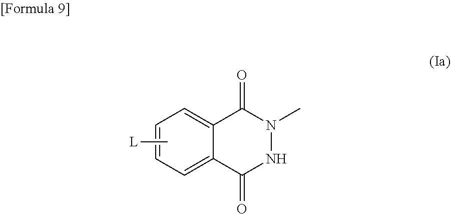

Here, L can be present at any position on the benzene ring, and is preferably present at the 6-position or the 7-position on hydrazide phthalate. As the compound represented by general formula (Ia), only one compound may be used, or a mixture of two or more compounds may be used. For example, a mixture of a compound in which L is present at the 6-position and a compound in which L is present at the 7-position may be used.

As the fluorescent dye, a fluorescent dye used in conventional Q-body, for example a fluorescent dye disclosed in the description of WO 2013/065314 can be used. Specific examples thereof include fluorescent dyes having rhodamine, coumarin, Cy, EvoBlue, oxazine, Carbopyronin, naphthalene, biphenyl, anthracene, phenenthrene, pyrene, carbazole, BODIPY (4,4-difluoro-4-bora-3a,4a-diaza-s-indasene) or the like as a basic skeleton, and derivatives of the fluorescent dyes. Specific examples of the fluorescent dye include CR110: carboxyrhodamine 110: Rhodamine Green (trade name), TAMRA: carbocytetremethlrhodamine: TMR, Carboxyrhodamine 6G: CR6G, ATTO655 (trade name), BODIPY FL (trade name): 4,4-difluoro-5,7-dimethyl-4-bora-3a,4a-diaza-s-indancene-3-propionic acid, BODIPY 493/503 (trade name): 4,4-difluoro-1,3,5,7-tetramethyl-4-bora-3a,4a-diaza-s-indancene-8-propionicacid, BODIPY R6G (trade name): 4,4-difluoro-5-(4-phenyl-1,3-butadienyl)-4-bora-3a,4a-diaza-s-indancene-3-propionic acid, BODIPY 558/568 (trade name): 4,4-difluoro-5-(2-thienyl)-4-bora-3a,4a-diaza-s-indancene-3-propionic acid, BODIPY 564/570 (trade name): 4,4-difluoro-5-styryl-4-bora-3a,4a-diaza-s-indancene-3-propionic acid, BODIPY 576/589 (trade name): 4,4-difluoro-5-(2-pyrrolyl)-4-bora-3a,4a-diaza-s-indancene-3-propionic acid, BODIPY 581/591 (trade name): 4,4-difluoro-5-(4-phenyl-1,3-butadienyl)-4-bora-3a,4a-diaza-s-indancene-3-propionic acid, BODIPY TMR (trade name), Cy3 (trade name), Cy3B (trade name), Cy3.5 (trade name), Cy5 (trade name), Cy5.5 (trade name), EvoBlue10 (trade name), EvoBlue30 (trade name), MR121, ATTO 390 (trade name), ATTO 425 (trade name), ATTO 465 (trade name), ATTO488 (trade name), ATTO 495 (trade name), ATTO 520 (trade name), ATTO 532 (trade name), ATTO Rho6G (trade name), ATTO 550 (trade name), ATTO 565 (trade name), ATTO Rho3B (trade name), ATTO Rho11 (trade name), ATTO Rho12 (trade name), ATTO Thio12 (trade name), ATTO 610 (trade name), ATTO 611X (trade name), ATTO 620 (trade name), ATTO Rho14 (trade name), ATTO 633 (trade name), ATTO 647 (trade name), ATTO 647N (trade name), ATTO 655 (trade name), ATTO OXa12 (trade name), ATTO 700 (trade name), ATTO 725 (trade name), ATTO 740 (trade name), Alexa Fluor 350 (trade name), Alexa Fluor 405 (trade name), Alexa Fluor 430 (trade name), Alexa Fluor 488 (trade name), Alexa Fluor 532 (trade name), Alexa Fluor 546 (trade name), Alexa Fluor 555 (trade name), Alexa Fluor 568 (trade name), Alexa Fluor 594 (trade name), Alexa Fluor 633 (trade name), Alexa Fluor 647 (trade name), Alexa Fluor 680 (trade name), Alexa Fluor 700 (trade name), Alexa Fluor 750 (trade name), Alexa Fluor 790 (trade name), Rhodamine Red-X (trade name), Texas Red-X (trade name), 5(6)-TAMRA-X (trade name), 5TAMRA (trade name) and SFX (trade name). Examples of particularly preferred fluorescent dyes among the above-mentioned fluorescent dyes include CR110 and TAMRA which are rhodamine-based fluorescent dyes, ATTO655 which is an oxazine-based fluorescent dye; ATTO655 which is an oxazine-based fluorescent dye; and BODIPY FL, BODIPY R6G, BODIPY 558/568, BODIPY 581/591 and BODIPY TMR which are BODIPY-based fluorescent dyes.

Normally, the fluorescent dye is bound to compounds represented by general formulas (I) to (VI) through groups for use in click reaction, such as an azide group. Therefore, it is preferable that the fluorescent dye have a structure which binds to an azide group, such as an ethynyl group or a cycloalkyne. More specifically, it is preferable that the fluorescent dye have dibenzylcyclooctine (DBCO) or the like. The fluorescent dye may be bound to a compound such as DBCO through a linker, or without a linker.

The antibody used is not particularly limited, and for example, rituximab which is an anti-CD20 antibody, or trastuzumab which is an anti-HER2 antibody can be used. The Q-body is intended to detect a low-molecular compound (WO 2013/065314), and the fluorescence-labeled antibody or antibody fragment of the present invention is intended to detect a low-molecular compound, so that an antibody against a low-molecular compound may be used. Examples of the low-molecular compound mentioned here include stimulant drugs and narcotic drugs such as amphetamine, methamphetamine, morphine, heroin and codeine, fungal toxins such as aflatoxin, sterigmatocystin, neosolaniol, nivalenol, fumonisin, ochratoxin and endophyte producing toxins, sex hormones such as testosterone and estradiol, additives illegally used in fodder, such as clenbuterol and ractopamine, hazardous substances such as PCB, gossypol, histamine, benzpyrene, melamine, acrylamide and dioxin, residual agricultural chemicals such as acetamiprid, imidacloprid, chlorfenapyr, malathion, carbaryl, clothianidin, triflumizole, chlorothalonil, Spinosad, Lannate, methamidophos and chlorpyrifos, and environmental hormones such as bisphenol A. Further, when the fluorescence-labeled antibody or antibody fragment of the present invention is used for diagnostic agents for diseases, an antibody against a substance serving as a marker for the disease can be used.

As the antibody fragment, a fragment of the above-described antibody can be used. The antibody fragment may be any fragment as long as a fluorescence dye bound to the fragment is quenched in the absence of an antigen and the quenching is cancelled in the presence of an antigen as with a full-length antibody. For the fluorescence dye to show such a change, the antibody fragment is normally required to have a light-chain variable region and a heavy-chain variable region, and therefore it is preferable that the antibody fragment contain these two regions. Specific examples of the antibody fragment containing a light-chain variable region and a heavy-chain variable region include Fab fragments, F(ab′)₂ fragments and scFvs (single-chain variable fragments).

For expanding a decrease in fluorescence intensity during the quenching of the fluorescent dye and an increase in fluorescence intensity during cancellation of the quenching, a quencher for the fluorescent dye, or FRET (fluorescence resonance energy transfer) can be utilized (R. Abe et al., Scientific Reports 4, 4640 (2014), WO 2013/065314).

Examples of the method using a quencher include methods for binding the quencher to the variable region of a chain different from the chain (light chain or heavy chain) to which the fluorescent dye binds. When the quencher is bound to such a position, a quenching effect is exhibited with the quencher going close to the fluorescent dye in the absence of an antigen, and a quenching effect is no longer exhibited with the quencher going away from the fluorescent dye in the presence of an antigen. This enables further expansion of a decrease in fluorescence intensity during the quenching of the fluorescent dye and an increase in fluorescence intensity during cancellation of the quenching.

The quencher used is not particularly limited as long as it exhibits a quenching effect on the fluorescent dye. Examples of the quencher include quenching dyes having NBD: 7-nitrobenzofurazan, DABCYL, BHQ, ATTO, QXL, QSY, Cy, Lowa Black, IRDYE or the like as a basic skeleton, and derivatives of the quenching dyes. Specific examples thereof include NBD, DABCYL, BHQ-1 (trade name), BHQ-2 (trade name), BHQ-3 (trade name), ATTO540Q (trade name), ATTO580Q (trade name), ATTO612Q (trade name), QXL490 (trade name), QXL520 (trade name), QXL570 (trade name), QXL610 (trade name), QXL670 (trade name), QXL680 (trade name), QSY-35 (trade name), QSY-7 (trade name), QSY-9 (trade name), QSY-21 (trade name), Cy5Q (trade name), Cy7Q (trade name), Lowa Black FQ (trade name), Lowa Black RQ (trade name) and IRDYE QC-1 (trade name). Of these, NBD is preferable. As a combination of the fluorescent dye and the quencher in the fluorescence-labeled antibody or antibody fragment of the present invention, a combination can be appropriately selected which ensures that the quencher effectively quenches the fluorescent dye in the absence of an antigen, and the quencher does not hinder light emission of the fluorescent dye in the presence of an antigen. Examples thereof include combinations of fluorescent dyes: TAMRA and NBD.

The binding of the quencher to the antibody or antibody fragment may be performed by a method obtained by modifying the method using a compound binding to an amino acid residue in the antibody or antibody fragment (e.g., a compound represented by any of general formulas (I) to (VIII)), i.e., a method using a compound in which a fluorescent dye contained in a compound binding to an amino acid residue in the antibody or antibody fragment is replaced with a quencher. Another method may be employed.

Examples of the method using FRET include a method for binding another fluorescent dye forming the FRET pair to the variable region of a chain different from the chain (light chain or heavy chain) to which the fluorescent dye binds.

When another fluorescent dye forming a FRET pair is bound to such a position, FRET occurs with the another fluorescent dye going close to the fluorescent dye in the absence of an antigen, and FRET no longer occurs with the another fluorescent dye going away from the fluorescent dye in the presence of an antigen. This enables further expansion of a decrease in fluorescence intensity during the quenching of the fluorescent dye and an increase in fluorescence intensity during cancellation of the quenching.

Examples of the fluorescent dyes forming a FRET pair include combinations of CR110 and TAMRA.

The binding of another fluorescent dye to the antibody or antibody fragment may be performed by the method using a compound binding to an amino acid residue in the antibody or antibody fragment (e.g., a compound represented by any of general formulas (I) to (VIII)). Another method may be employed.

The fluorescent-labeled antibody or antibody fragment of the present invention has the following advantages.

1) A preparation, for which a preparation method has been established, such as an antibody drug, can be formed into a Q-body because tyrosine residues selectively present in a complementarity determining region of the antibody can be labeled efficiently (by almost 100%).
2) Since synthesis can be performed by a method based on chemical reaction, and a genetic engineering method is not required, high-volume synthesis is possible. 3) It is possible to form a polyclonal antibody into a Q-body, which has been heretofore difficult.

### (C) Diagnostic Agent

The diagnostic agent for disease according to the present invention contains the fluorescence-labeled antibody or antibody fragment of the present invention, which is an antibody or antibody fragment against a substance serving as a marker for disease.

Examples of the antibody against a substance serving as a marker for disease include rituximab and trastuzumab described above. Rituximab is an antibody against CD20 which is a marker for malignant lymphomas, and trastuzumab is an antibody against HER2 which is a marker for breast cancer, esophagus cancer and stomach cancer. In addition to these antibodies, many antibodies against substances serving as markers for disease are known, and most of such antibodies are commercially available. In the diagnostic agent for disease according to the present invention, such known antibodies and commercially available antibodies can be used. Specific examples of the antibody against a substance serving as a marker for disease include antibodies against EGFR serving as a marker for lung cancer and the like, antibodies against α-fetoprotein (AFP) serving as a marker for liver cancer, antibodies against calcitonin serving as a marker for thyroid gland medullary cancer, antibodies against CA15-3 serving as a marker for breast cancer, antibodies against CA19-9 serving as a marker for bile duct cancer, antibodies against CD45 serving as a marker for leukemia, antibodies against Myo D1 serving as a marker for rhabdomyosarcoma, antibodies against PLAP serving as a marker for embryonic cancer, antibodies against PSA serving as a marker for prostate cancer, antibodies against TTF1 serving as a marker for thyroid gland cancer, and antibodies against amyloid β and tau protein serving as markers for Alzheimer's disease.

The diagnostic agent for disease according to the present invention can be used as an extracorporeal diagnostic agent for examining a marker substance in collected samples from organisms such as humans (e.g., blood, spinal fluid, saliva, urine, feces and skin). The diagnostic agent can also be used as an intracorporeal diagnostic agent which is administered to organisms such as humans to examine a marker substance present in organs, tissues or cells in the organisms.

### (D) Method for Producing Fluorescent-Labeled Antibody or Antibody Fragment

The method for producing a fluorescence-labeled antibody or antibody fragment according to the present invention includes steps (1) and (2).

In step (1), a compound which binds to an amino acid residue in the antibody or antibody fragment and contains a functional group for use in click reaction is reacted with the antibody or antibody fragment to bind the compound to an amino acid residue in the antibody or antibody fragment.

Examples of the compound binding to an amino acid residue in an antibody or antibody fragment include compounds represented by general formulas (Ib) to (VIIIb).

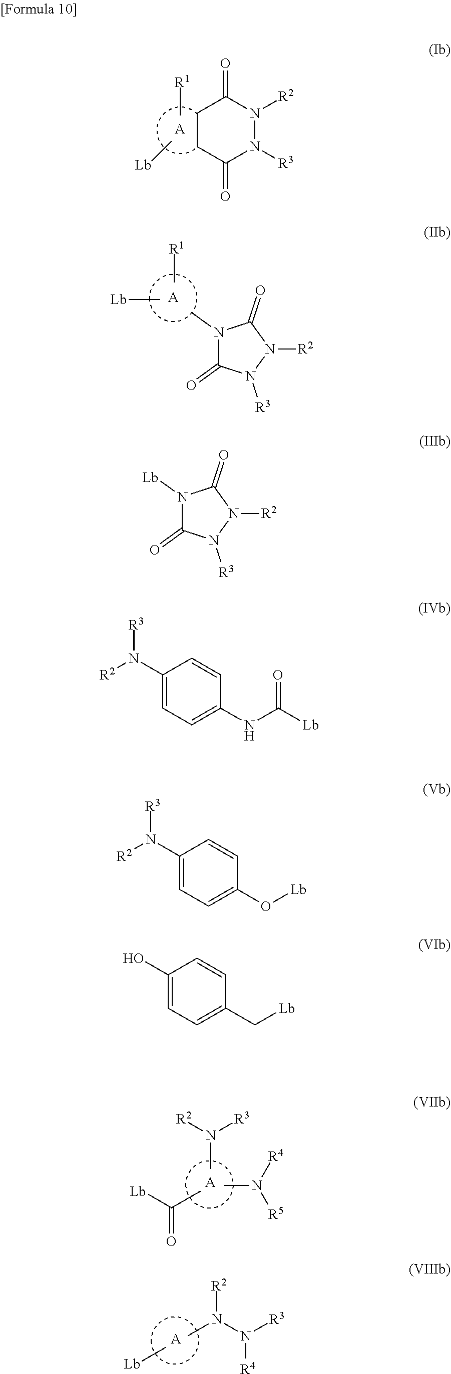

A, R¹, R², R³, R⁴ and R⁵ in general formulas (Ib) to (VIIIb) have the same meaning as A, R¹, R², R³, R⁴ and R⁵ in general formulas (I) to (VIII).

In general formulas (Ib) to (VIIIb), Lb represents a linker having a functional group for use in click reaction at the end. Lb can be present at any position on A when Lb is present on A. The linker may have any structure as long as the structure does not cause the functional group for use in click reaction to lose its function, and the linker is preferably an alkylene group. One or more —CH₂— in the alkylene group may be substituted with —O—, —S—, —NH— or —CO—. The number of carbon atoms in the alkylene group is not particularly limited, and is preferably 5 to 20, more preferably 5 to 15. When —CH₂— in the alkylene group is substituted with another group, the other group is considered to contribute to the “number of carbon atoms in the alkylene group” as a group having one carbon atom.

The compounds binding to an amino acid residue in the antibody or antibody fragment are not limited to compounds represented by general formulas (Ib) to (VIIIb) described above, and include, for example, the following compounds.

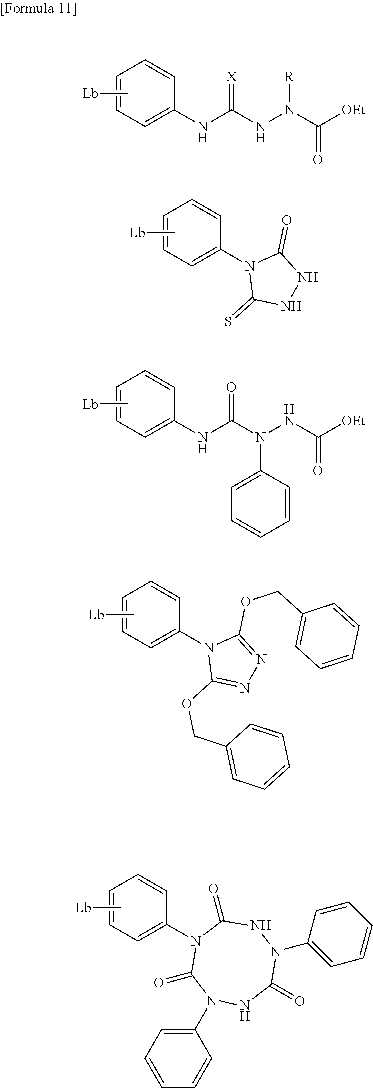

In the compounds, Lb has the same meaning of Lb in general formulas (Ib) to (VIIIb), X represents an oxygen atom or a sulfur atom, R represents a hydrogen atom or a methyl group when X represents an oxygen atom, and R represents a hydrogen atom when X represents a sulfur atom.

Among the compounds binding to an amino acid residue in the antibody or antibody fragment, compounds binding to a tyrosine residue in the antibody or antibody fragment are preferable. Examples of the compounds include compounds represented by general formulas (Ib) to (VIb).

Among the compounds represented by general formulas (Ib) to (VIb), compounds represented by general formula (Ib) are preferable to use. Among the compounds represented by general formula (Ib), compounds represented by general formula (Iab) are preferable to use.

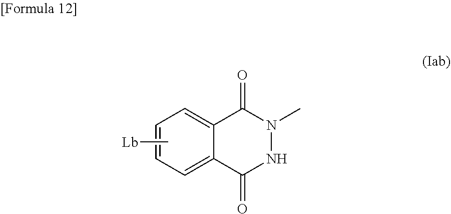

Here, Lb can be present at any position on the benzene ring, and is preferably present at the 6-position or the 7-position on hydrazide phthalate. As the compound represented by general formula (Iab), only one compound may be used, or a mixture of two or more compounds may be used. For example, a mixture of a compound in which Lb is present at the 6-position and a compound in which Lb is present at the 7-position may be used.

The compound binding to an amino acid residue in an antibody or antibody fragment is reacted with the antibody or antibody fragment normally in the presence of a catalyst such as HRP (horseradish peroxidase) and an oxidizing agent such as hydrogen peroxide, and the reaction may be carried out electrochemically without the use of such a catalyst and oxidizing agent.

### (a) Reaction Using Catalyst and Oxidizing Agent

The amount of the compound binding to an amino acid residue in an antibody or antibody fragment, which is used for the reaction, is not particularly limited, and is normally 1 to 1,000 mol, preferably 10 to 100 mol, per mol of the antibody or antibody fragment when the compound represented by general formula (Ib) is used.

The solvent used for the reaction is not particularly limited as long as it does not hinder the reaction, and for example, water, acetonitrile, methanol, ethanol, dimethyl sulfoxide or the like can be used.

The pH during the reaction is not particularly limited, and is normally 3 to 11, preferably 6 to 8.

The reaction temperature is not particularly limited, and is normally 4 to 50° C., preferably 10 to 30° C.

The reaction time is not particularly limited, and is normally 5 to 240 minutes, preferably 30 to 60 minutes.

When HRP is used as a catalyst, the amount thereof is not particularly limited, and is normally 0.001 to 10 mol, preferably 0.001 to 0.005 mol, per mol of the antibody or antibody fragment.

When hydrogen peroxide is used as an oxidizing agent, the amount thereof is not particularly limited, and is normally 1 to 100 mol, preferably 1 to 10 mol, per mol of the antibody or antibody fragment.

### (b) Electrochemical Reaction

The compound binding to an amino acid residue in an antibody or antibody fragment may be electrochemically reacted with the antibody or antibody fragment by applying a voltage to a solution containing the compound and the antibody or antibody fragment.

The solvent used is not particularly limited as long as it is electrically conductive, and does not adversely affect the antibody or antibody fragment. For example, an electrolytic solution such as a Tris-HCl buffer solution or a phosphate buffer solution (e.g., potassium phosphate buffer solution) can be used as the solvent. The pH of the solution is not particularly limited, and is preferably 5 to 9, more preferably 6 to 8.

The concentration of the compound binding to an amino acid residue in an antibody or antibody fragment in the solution is appropriately set according to the type of the compound used, and is preferably 50 to 1,000 μM, more preferably 100 to 500 μM, when the compound represented by general formula (Ib) is used.

The concentration of the antibody or antibody fragment in the solution is not particularly limited, and is preferably 0.1 to 100 μM, more preferably 1 to 10 μM.

The application of a voltage can be performed by, for example, putting a solution into an electrolytic bath, inserting electrodes in the solution, and applying the voltage between the electrodes from the outside. Such application of a voltage can be performed with a commercially bulk electrolytic cell and the like. The electrodes may be the same as those that are used in general electrochemical reaction, and for example, carbon electrodes, platinum electrodes, silver electrodes, copper electrodes or the like can be used as the electrodes. The carbon electrode is preferably a porous carbon electrode having a large surface area.

The electrolytic process may be either a constant current process or a constant voltage process, and is preferably a constant voltage process. When a constant voltage process is employed, it is preferable to use a commercially available potentiostat.

When a voltage is applied by a constant voltage process, the voltage is not particularly limited, and is preferably 0.1 to 5.0 V, more preferably 0.1 to 2.0 V.

The voltage application time (cumulative time) is not particularly limited, and is preferably 1 second to 120 minutes, more preferably 1 to 60 minutes.

After completion of the reaction (a) or (b), substances other than intended products (e.g., an antibody or antibody fragment bound to a compound represented by any of general formulas (Ib) to (VIIIb)) may be removed by chromatography or the like.

In step (2), the fluorescent dye is reacted with a functional group for use in click reaction in the compound represented by any of general formulas (Ib) to (VIb), to bind the fluorescent dye to the functional group.

Step (2) may be carried out continuously or non-continuously from step (1).

The reaction of step (2) is a well-known reaction as click reaction, and persons skilled in the art can appropriately set the conditions of the reaction.

The same fluorescent dye as described in “(B) fluorescence-labeled antibody or antibody fragment” can be used.

The amount of the fluorescent dye used for the reaction is not particularly limited, and is normally 1 to 100 mol, preferably 1 to 10 mol, based on 1 mol of the antibody or antibody fragment, when DBCO-TAMRA is used as the fluorescent dye.

The solvent used for the reaction is not particularly limited as long as it does not hinder the reaction, and for example, water, acetonitrile, methanol, ethanol, dimethyl sulfoxide or the like can be used.

The pH during the reaction is not particularly limited, and is normally 3 to 11, preferably 6 to 8.

The reaction temperature is not particularly limited, and is normally 4 to 50° C., preferably 25 to 40° C.

The reaction time is not particularly limited, and is normally 5 to 720 minutes, preferably 5 to 60 minutes.

### EXAMPLES

Hereinafter, the present invention will be described in more detail by way of Examples, which should not be construed as limiting the present invention.

### [Example 1] Cancellation of Quenching by Denaturant

Rituximab which is an anti-human CD 20 antibody (CHUGAI PHARMACEUTICAL CO., LTD.) was dissolved in TBS Tris buffered saline (100 mM Tris-HCl, 150 mM NaCl, pH 7.4) at a concentration of 5 μM, and HRP and NMeL uminol-N₃ having the following structure were added thereto to final concentrations of 45 nM and 300 μM, respectively.

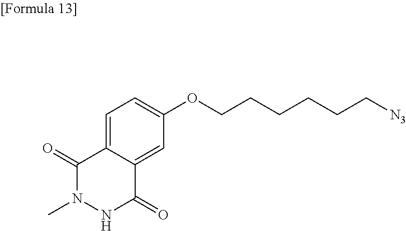 [SMILES: Cn1[nH]c(=O)c2cc(OCCCCCCN=[N+]=[N-])ccc2c1=O]

H₂O₂ was added to a final concentration of 25 μM, and the mixture was stirred, and then left standing at room temperature for 1 hour. NMeLuminol-N₃ which was not bound to rituximab was removed with a Sephadex-G25 column.

To N₃ group-modified rituximab was added DBCO-PEG₄-TAMRA (Aldrich) to a final concentration of 10 μM, and the mixture was reacted at 37° C. for 30 minutes. Low-molecular compounds were removed with a Sephadex-G25 column.

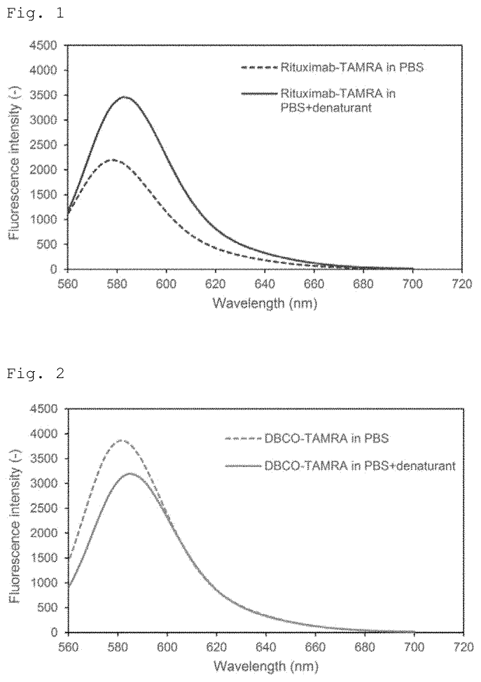

The final concentration of rituximab was adjusted to 50 nM, and the fluorescence was measured in the presence and absence of a denaturant using a fluorospectrophotometer (FP-8300 from JASCO Corporation) (FIG. 1). As the denaturant, guanidine was used, and the concentration was set to 7 M. For comparison, the fluorescence of DBCO-PEG₄-TAMRA which was not bound to rituximab was measured (FIG. 2).

As shown in the figures, the fluorescence intensity of DBCO-PEG₄-TAMRA was decreased by being bound to rituximab. On the other hand, the fluorescence intensity of DBCO-PEG₄-TAMRA bound to rituximab was increased by adding the denaturant.

### [Example 2] Cancellation of Quenching by Lysate of Cells Expressing CD20

Rituximab bound to DBCO-PEG₄-TAMRA was prepared in the same manner as in Example 1. The final concentration of rituximab was adjusted to 50 nM, and the fluorescence was measured in the presence of a cell lysate at each concentration using a fluorospectrophotometer (FP-8300 from JASCO Corporation). As the cell lysate, a lysate of SU-DHL-4 cells (ATCC) which are cells expressing an antigen (CD20) (left in FIG. 3) and a lysate of SK-BR-3 cells (ATCC) which are cells that do not express an antigen (right in FIG. 3) were used.

As shown in the figure, the fluorescence intensity hardly increased when the lysate of SK-BR-3 cells was added, whereas the fluorescence intensity increased with a concentration of the lysate when the lysate of SU-DHL-4-cells was added.

### [Example 3] Effect of Linker on Cancellation of Quenching

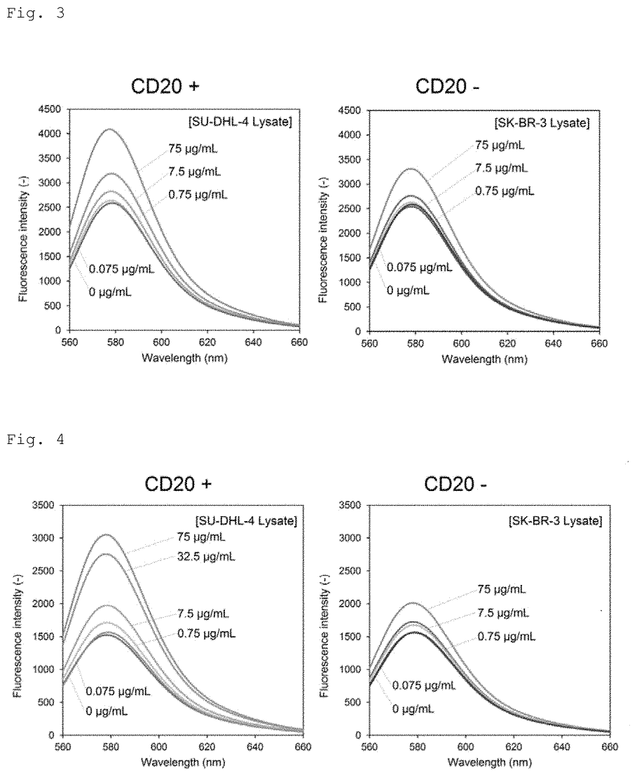

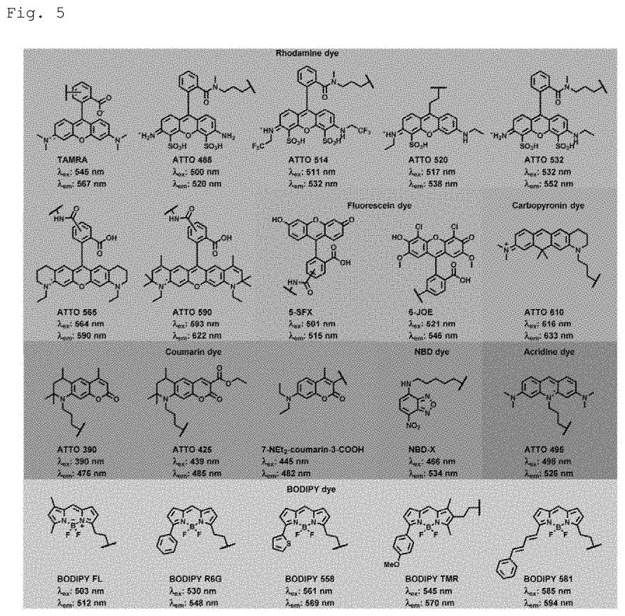

The fluorescence was measured in the presence of a lysate of SU-DHL-4 cells (left in FIG. 4) or a lysate of SK-BR-3 cells (right in FIG. 4) using a fluorospectrophotometer (FP-8300 from JASCO Corporation) as in Example 2 except that DBCO-TAMRA was used instead of DBCO-PEG₄-TAMRA. In DBCO-TAMRA, PEG₄, which is a linker for DBCO-PEG₄-TAMRA is removed, and DBCO and TAMRA are in proximity.

Comparison between FIGS. 3 and 4 indicates that use of DBCO-TAMRA leads to a larger increase in fluorescence intensity during addition of the lysate of antigen expressing cells, and the shorter the linker between DBCO and TAMRA, the more suitable for detection of an antigen.

### [Example 3] Preparation of Modified Antibodies to which Various Fluorescent Dyes are Bound

A 100 mM DMF solution of DBCO-amine (Aldrich) (2 equivalents), N,N-diisopropylethylamine (2 equivalents) and a NHS-binding fluorescent dye (1 equivalent) were reacted in DMF for 1 hour, the reaction mixture was added to N₃ group-modified rituximab or N₃ group-modified trastuzumab to a final concentration of 10 μM in terms of DBCO-binding fluorescent dye molecules (1 equivalent), and the mixture was reacted at 37° C. for 30 minutes. Low-molecular compounds were removed with a Sephadex-G25 column. The N₃ group-modified trastuzumab was prepared by the same procedure as in the case of the N₃ group-modified rituximab (Example 1).

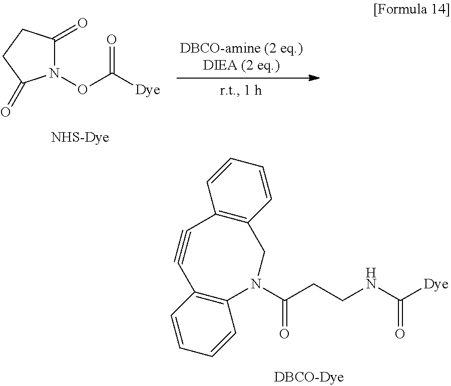

A total of 20 fluorescent dyes from Rhodamine dyes including TAMRA, Fluorescein dyes, Carbopyronin dyes, Coumarin dyes, NBD dyes, Acridine dyes and BODIPY dyes were used in studies (FIG. 5). For the antibodies modified with these fluorescent dyes, the fluorescence intensity in the quenching state (Quenching) and the fluorescence intensity in the de-quenching state (De-Quenching) were determined, and the ratio of both the intensities (De-Quenching intensity/Quenching intensity) was defined as a response ratio (Res. Ratio). The response ratios of the antibodies modified with the fluorescent dyes are shown in the table below. The de-quenching state was generated by adding a denaturant rather than adding an antigen.

TABLE 1

| | | |
|---|---|---|
| Antibody | Dye | Res. Ratio |

a) Denaturation (Rituximab)

| | | |
|---|---|---|
| Rituximab | TAMRA | 2.2 |
| (7.5 μg/mL) | ATTO 488 | 2.1 |
| | ATTO 514 | 1.0 |
| | ATTO 520 | 2.4 |
| | ATTO 532 | 1.2 |
| | ATTO 565 | 2.1 |
| | ATTO 590 | 2.6 |
| | 5-SFX | 0.4 |
| | 6-JOE | 1.1 |
| | ATTO 610 | 2.1 |
| | ATTO 390 | 1.0 |
| | ATTO 425 | 2.5 |
| | 7-NEt₂-coumarin-3-COOH | 1.7 |
| | NBD-X | 1.4 |
| | ATTO 495 | 2.9 |
| | BODIPY FL | 3.3 |
| | BODIPY R6G | 2.1 |
| | BODIPY 558 | 2.3 |
| | BODIPY TMR | 2.6 |
| | BODIPY 581 | 2.5 |

b) Denaturation (Trastuzumab)

| | | |
|---|---|---|
| Trastuzumab | TAMRA | 1.5 |
| (7.5 μg/mL) | ATTO 488 | 1.4 |
| | ATTO 514 | 0.9 |
| | ATTO 520 | 2.6 |
| | ATTO 532 | 0.9 |
| | ATTO 565 | 1.7 |
| | ATTO 590 | 1.4 |
| | 5-SFX | 0.5 |
| | 6-JOE | 0.9 |
| | ATTO 610 | 2.0 |
| | ATTO 390 | 1.2 |
| | ATTO 425 | 2.2 |
| | 7-NEt₂-coumarin-3-COOH | 1.4 |
| | NBD-X | 1.4 |
| | ATTO 495 | 2.9 |
| | BODIPY FL | 3.0 |
| | BODIPY R6G | 1.6 |
| | BODIPY 558 | 2.7 |
| | BODIPY TMR | 2.1 |
| | BODIPY 581 | 2.2 |

When rituximab was used, TAMRA, ATTO 488, ATTO 520, ATTO 565 and ATTO 590 from Rhodamine dyes, ATTO 610 from Carbopyronin dyes, ATTO 425 from Coumarin dyes, ATTO 495 from Acridine dyes, and BODIPY FL, BODIPY R6G, BODIPY 558, BODIPY TMR and BODIPY 581 from BODIPY dyes produced a good response ratio. On the other hand, when trastuzumab was used, ATTO 520 from Rhodamine dyes, ATTO 610 from Carbopyronin dyes, ATTO 425 from Coumarin dyes, and BODIPY FL, BODIPY R6G, BODIPY 558, BODIPY TMR and BODIPY 581 from BODIPY dyes produced a good response ratio.

### [Example 4] Method for Electrochemically Preparing Modified Rituximab

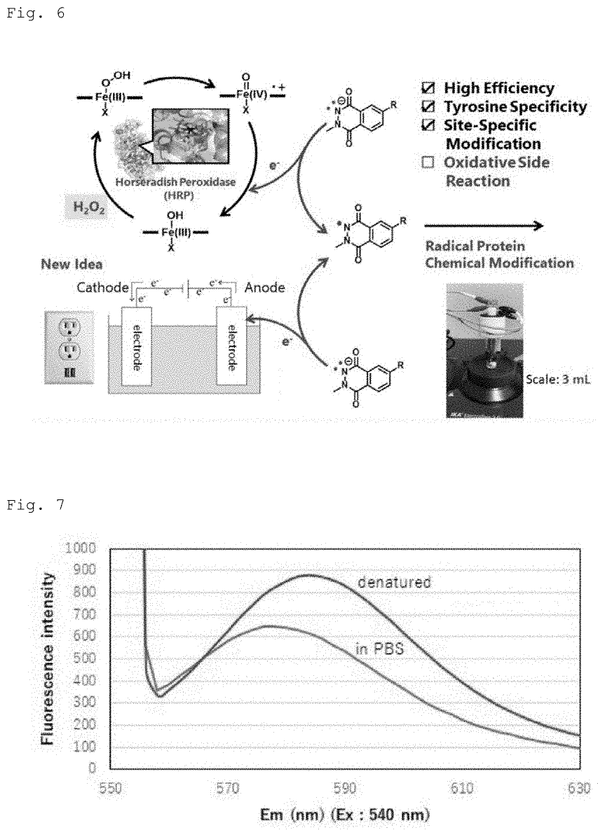

Rituximab which is an anti-human CD20 antibody (CHUGAI PHARMACEUTICAL CO., LTD.) was dissolved in Tris buffer (50 mM Tris-HCl, pH 7.4) at a concentration of 5 μM, and NMeLuminol-N₃ was added to a concentration of 300 μM. An electrochemical reaction apparatus (positive electrode and counter electrode: RVC electrodes (IKA), reference electrode: Ag/AgCl (IKA)) as shown in FIG. 6 was used. The mixture was stirred at a speed of 400 rpm, and a voltage was applied with HSV-110 (HOKUTO DENKO CORPORATION). After the voltage was applied for 30 minutes at a constant voltage of 0.7 V, and low-molecular compounds were removed with a Sephadex-G25 column.

To 5 μM of the N₃ group-modified rituximab was added DBCO-TAMRA as a fluorescent dye to a final concentration of 10 μM, and the mixture was reacted at 37° C. for 30 minutes. Low-molecular compounds were removed with a Sephadex-G25 column.

FIG. 7 shows fluorescence spectra in the quenching state (in the absence of the denaturant) and in the de-quenching state (in the presence of the denaturant) of the TAMRA-labeled rituximab. As shown in FIG. 7, the fluorescence-labeled rituximab prepared by an electrochemical method was de-quenched by adding the denaturant as in the case of the fluorescence-labeled rituximab prepared with the use of HRP and H₂O₂.

All the publications, patents and patent applications cited in the present description are incorporated in their entirety by reference.

### INDUSTRIAL APPLICABILITY

The fluorescence-labeled antibody or antibody fragment of the present invention can be used for diagnostic agents for various diseases, and are therefore applicable in industrial fields related to production of such diagnostic agents.

1-18. (canceled)

19. A fluorescence-labeled antibody or antibody fragment which is an antibody or antibody fragment labeled with a fluorescent dye, the fluorescent dye having the following properties (1) and (2):
(1) the fluorescent dye is quenched in the absence of an antigen and the quenching of the fluorescent dye is cancelled in the presence of an antigen; and
(2) the fluorescent dye is contained in a compound binding to an amino acid residue in the antibody or antibody fragment, and the compound is a compound represented by any of general formulas (I) to (VIII): 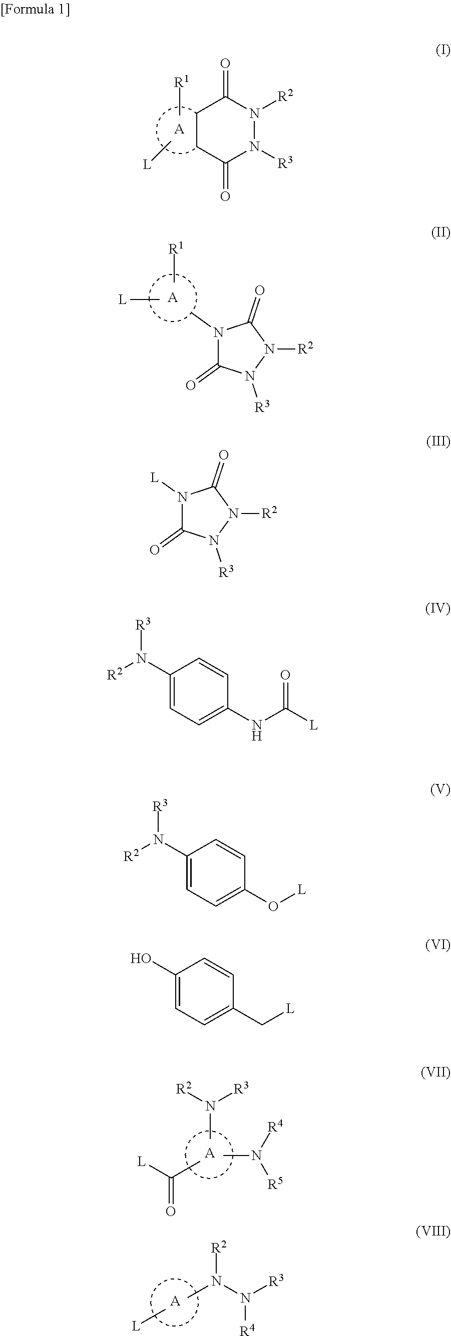
wherein A represents a conjugated ring, L represents a linker having a fluorescent dye at an end (provided that L can be present at any position on A when L is present on A), R¹ represents a hydrogen atom, or represents one or two amino groups, acetamide groups, hydroxy groups, alkyl groups or alkoxy groups each present at any position on A, and R², R³, R⁴ and R⁵ each represent a hydrogen atom, an alkyl group, or an aromatic group optionally having a substituent.

20. The fluorescence-labeled antibody or antibody fragment according to claim 19, wherein the compound binding to an amino acid residue in the antibody or antibody fragment is a compound binding to a tyrosine residue in the antibody or antibody fragment.

21. The fluorescence-labeled antibody or antibody fragment according to claim 20, wherein the compound binding to a tyrosine residue in the antibody or antibody fragment is a compound represented by any of general formulas (I) to (VI): 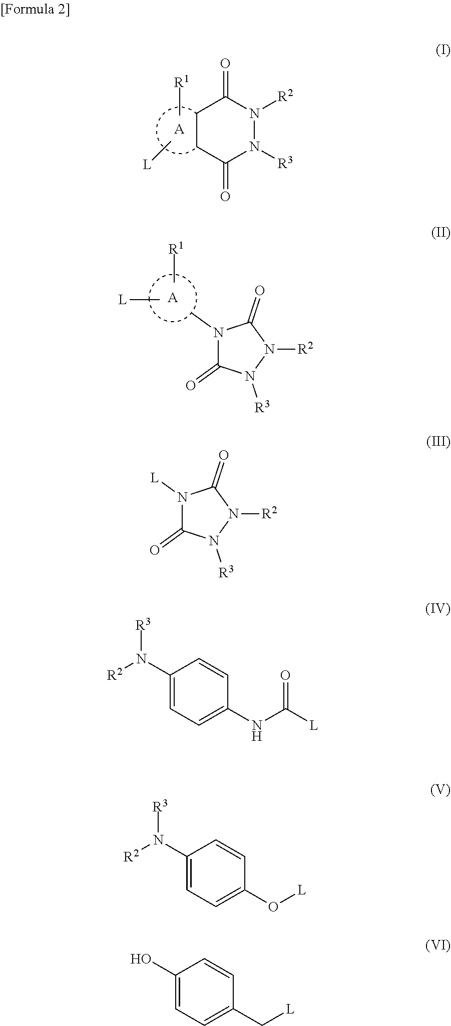
wherein A represents a conjugated ring, L represents a linker having a fluorescent dye at an end (provide that L can be present at any position on A when L is present on A), le represents a hydrogen atom, or represents one or two amino groups, acetamide groups, hydroxy groups, alkyl groups or alkoxy groups each present at any position on A, and R² and R³ each represent a hydrogen atom, an alkyl group, or an aromatic group optionally having a substituent.

22. The fluorescence-labeled antibody or antibody fragment according to claim 20, wherein the compound binding to a tyrosine residue in the antibody or antibody fragment is a compound represented by general formula (Ia): 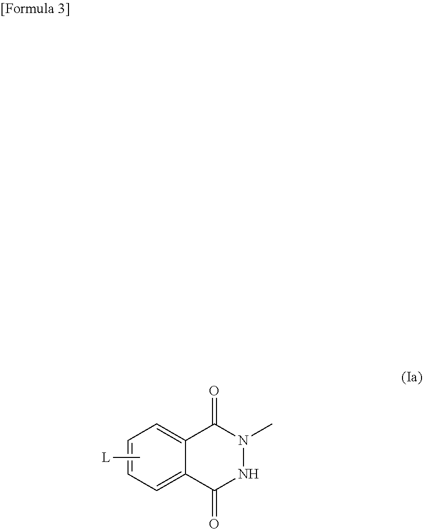
wherein L represents a linker which is present at any position on the benzene ring and has a fluorescent dye at an end.

23. The fluorescence-labeled antibody or antibody fragment according to claim 19, wherein the fluorescent dye is a fluorescent dye having 4,4-difluoro-4-bora-3a,4a-diaza-s-indacene as a basic skeleton.

24. The fluorescence-labeled antibody or antibody fragment according to claim 19, wherein the antibody or antibody fragment contains a light chain and a heavy chain, and the fluorescent dye binds to a variable region of the light chain or the heavy chain.

25. The fluorescence-labeled antibody or antibody fragment according to claim 24, wherein the antibody or antibody fragment binds to a quencher for the fluorescent dye, and the quencher binds to a variable region of a chain different from the chain to which the fluorescent dye binds.

26. The fluorescence-labeled antibody or antibody fragment according to claim 24, wherein the antibody or antibody fragment binds to another fluorescent dye forming a FRET pair with the fluorescent dye, and the other fluorescent dye binds to a variable region of a chain different from the chain to which the fluorescent dye binds.

27. The fluorescence-labeled antibody or antibody fragment according to claim 19, wherein the antibody or antibody fragment is an antibody or antibody fragment against a substance serving as a marker for disease.

28. A diagnostic agent for disease, comprising the fluorescence-labeled antibody or antibody fragment according to claim 27.

29. A method for producing a fluorescence-labeled antibody or antibody fragment, the method comprising the following steps (1) and (2):
(1) reacting a compound binding to an amino acid residue in an antibody or antibody fragment, the compound containing a functional group for use in click reaction, with the antibody or antibody fragment to bind the compound to the amino acid residue in the antibody or antibody fragment; and
(2) reacting a fluorescent dye with the functional group for use in click reaction in the compound binding to an amino acid residue in an antibody or antibody fragment to bind the fluorescent dye to the functional group,
the fluorescence-labeled antibody or antibody fragment being labeled with a fluorescent dye having the following property (A):
(A) the fluorescent dye is quenched in the absence of an antigen and the quenching of the fluorescent dye is cancelled in the presence of an antigen.

30. The method for producing a fluorescence-labeled antibody or antibody fragment according to claim 29, wherein the compound binding to an amino acid residue in an antibody or antibody fragment is a compound represented by any of general formulas (Ib) to (VIIIb): 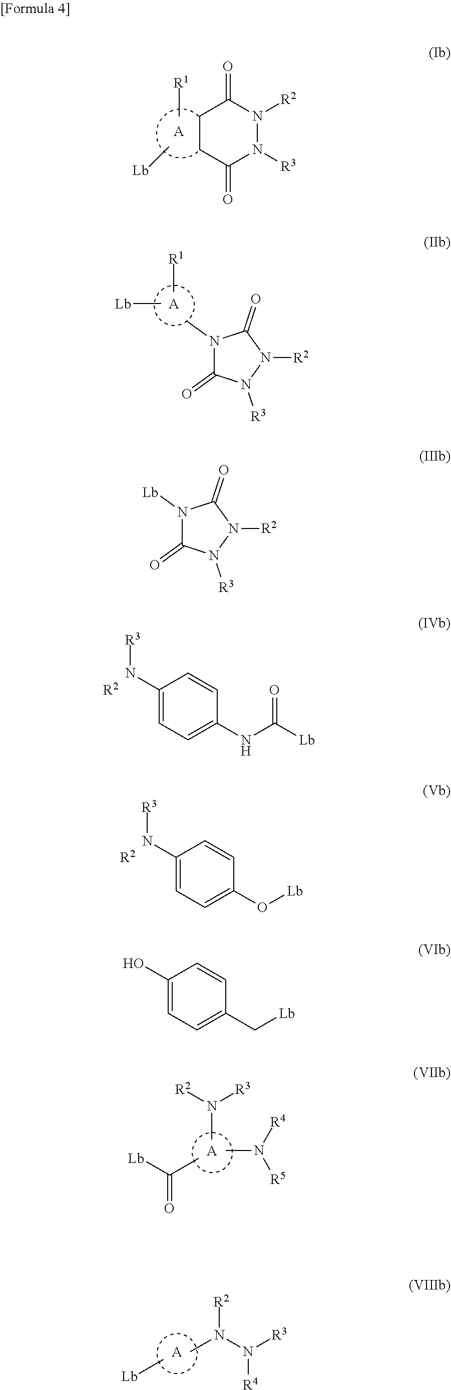
wherein A represents a conjugated ring, Lb represents a linker having a functional group for use in click reaction at an end (provide that Lb can be present at any position on A when Lb is present on A), R¹ represents a hydrogen atom, or represents one or two amino groups, acetamide groups, hydroxy groups, alkyl groups or alkoxy groups each present at any position on A, and R², R³, R⁴ and R⁵ each represent a hydrogen atom, an alkyl group, or an aromatic group optionally having a substituent.

31. The method for producing a fluorescence-labeled antibody or antibody fragment according to claim 29, wherein the compound binding to an amino acid residue in an antibody or antibody fragment is a compound binding to a tyrosine residue in the antibody or antibody fragment.

32. The method for producing a fluorescence-labeled antibody or antibody fragment according to claim 31, wherein the compound binding to a tyrosine residue in the antibody or antibody fragment is a compound represented by any of general formulas (Ib) to (VIb): 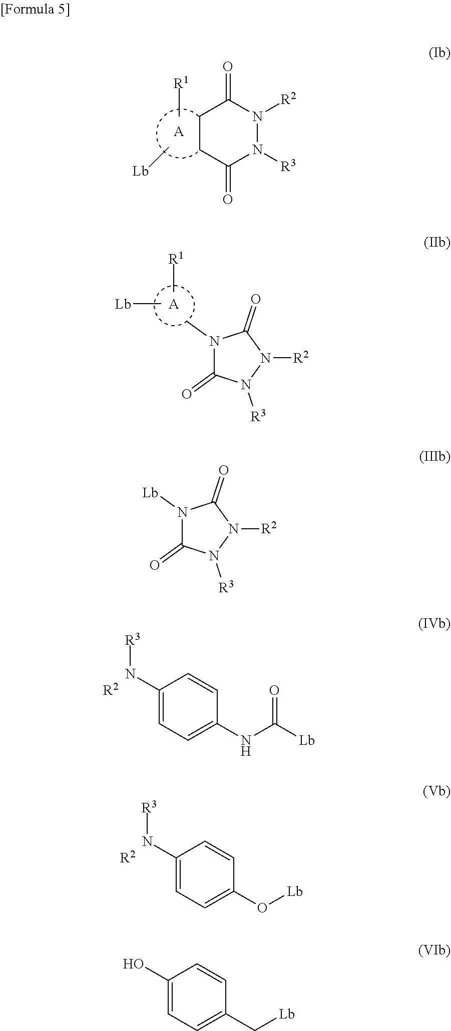
wherein A represents a conjugated ring, Lb represents a linker having a functional group for use in click reaction at an end (provide that Lb can be present at any position on A when Lb is present on A), R¹ represents a hydrogen atom, or represents one or two amino groups, acetamide groups, hydroxy groups, alkyl groups or alkoxy groups each present at any position on A, and R² and R³ each represent a hydrogen atom, an alkyl group, or an aromatic group optionally having a substituent.

33. The method for producing a fluorescence-labeled antibody or antibody fragment according to claim 31, wherein the compound binding to a tyrosine residue in the antibody or antibody fragment is a compound represented by general formula (Iab): 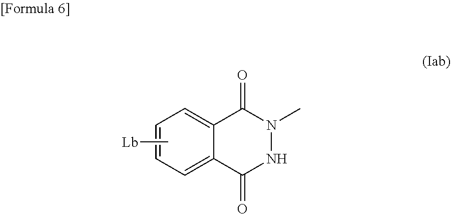
wherein Lb represents a linker which is present at any position on the benzene ring and has a functional group for use in click reaction at an end.

34. The method for producing a fluorescence-labeled antibody or antibody fragment according to claim 29, wherein the fluorescent dye is a fluorescent dye having 4,4-difluoro-4-bora-3a,4a-diaza-s-indacene as a basic skeleton.

35. The method for producing a fluorescence-labeled antibody or antibody fragment according to claim 29, wherein step (1) is a step of applying a voltage to a solution containing the compound binding to an amino acid residue in an antibody or antibody fragment and the antibody or antibody fragment.
